(19) United States
(12) Patent Application Publication
(10) Pub. No.: US 2006/0064059 A1
(43) Pub. Date: Mar. 23, 2006

(54) Treatment of infarct expansion by partially occluding vena cava

(76) Inventors: Mark Gelfand, New York, NY (US); Howard Levin, Teaneck, NJ (US)

Correspondence Address: NIXON & VANDERHYE, PC, 901 NORTH GLEBE ROAD, 11TH FLOOR, ARLINGTON, VA, 22203, US

(21) Appl. No.: 11/230,649

(22) Filed: Sep. 21, 2005

Related U.S. Application Data
(60) Provisional application No. 60/611,282, filed on Sep. 21, 2004.

(51) Int. Cl. A61M 29/00; A61M 31/00

(52) U.S. Cl. 604/103.06; 604/500

(57) ABSTRACT

A method and apparatus for prevention and reduction of myocardial infarct size and/or expansion and heart remodeling by partial, controllable and reversible obstruction of the venous blood flow to the heart. As a result, the ventricular wall stress and dilation are reduced. Blood flow is maintained at a safe level for the duration of treatment. The apparatus consists of a catheter with an occlusion balloon and a control and monitoring system.

### RELATED APPLICATION

This application claims the benefit of the Sep. 21, 2004, filing date of U.S. Provisional Application Ser. No. 60/611,282, the entirety of which is incorporated by reference.

### BACKGROUND OF INVENTION

A. Field of the Invention

This invention relates to a method for preventing expansion of the myocardial infarct size following a heart attack. It also relates to the reduction of the volume of the heart by partial occlusion of the vena cava.

B. Background of the Invention

A Myocardial Infarction (MI), or heart attack, starts when a coronary artery suddenly becomes occluded and can no longer supply blood to the myocardial tissue. Within seconds of coronary artery occlusion, the under-perfused myocardial cells no longer contract, leading to abnormal ventricular wall motion and reduced blood flow to the body. If the occlusion lasts for a long enough period of time (minutes to hours), the myocardial tissue that is no longer receiving adequate blood flow dies and causes biochemical and structural changes in that tissue. Both the abnormal ventricular wall motion of the and changes in the composition of the ventricular wall muscle create high stresses within the infarcted muscle area as well as the areas surrounding the infarct, leading to further depression of ventricular function. The further depressed ventricular function results in an increase in contractility (the force generated by or “squeeze” of the heart muscle). The increased contractility temporarily regains lost blood flow to the body but at the cost of increased oxygen demand by and increased wall stress in the heart muscle. These increased stresses lead to the occurrence of infarct expansion and ventricular remodeling at the junction between the infarcted tissue and the adjacent heart muscle with low but still minimally adequate blood flow which is still at risk for becoming infarcted if due to increase oxygen demand from increased wall stress. If this occurs, the expansion of the infarcted areas results in a ever-increasing wave of dysfunctional tissue spreading out from the original myocardial infarct region.

Left ventricular remodeling is defined as changes in shape and size of the Left Ventricle (LV) that can follow a MI. The process of LV enlargement can be influenced by three independent factors, that is, infarct size, infarct healing and LV wall stress. The process is a continuum, beginning in the acute period (during and in the hours to days after the coronary artery occlusion) and continuing through and beyond the late convalescent period (days to weeks).

The area of actual destruction, or necrosis, of myocardial tissue is called the infarct size. The infarct size is determined by the balanced between metabolic demand and oxygen supply during and after the period of coronary artery occlusion. Thus, methods that lower the oxygen demand or increase the oxygen supply during this period will limit the size of the damage. Infarct healing is a complex process of biochemical and physical changes that occurs to replace or compensate for the loss of muscle cells from the infarction. Some of these changes directly affect the structure of the collagen in the heart muscle, or the structural component that helps the heart maintain its size and shape. During the early period after MI, the collagen and other tissues within the infarcted and adjacent regions are particularly vulnerable to distorting forces caused by increased wall stress. This period of remodeling is called infarct expansion. The infarct expansion phase of remodeling starts on the first day of MI (likely as soon as hours after the beginning of the MI) and lasts up to 14 days. Once healed, the infarcted tissue or “scar” itself is relatively non distensible and much more resistant to further deformation. Therefore, late enlargement is due to complex alterations in LV architecture involving both infarcted and non-infarcted zones. This late chamber enlargement is associated with lengthening of the contractile regions rather than progressive infarct expansion. Post infarction LV aneurysm (a bulging out of a thin, weak area of the ventricular wall) represents an extreme example of adverse remodeling that leads to progressive deterioration of function that can lead to symptoms and signs of congestive heart failure.

C. Prior Art Treatments

The most effective treatments for MI are acute and can be only implemented immediately after the occlusion of the coronary vessel. The newest approaches include aggressive efforts to restore patency to occluded vessels broadly called reperfusion therapies. This is accomplished through thrombolytic therapy (with drugs that dissolve the thrombus) or increasingly with primary angioplasty and stents. Reopening the occluded artery within hours of the initial occlusion can decrease tissue death, and thereby decrease the total magnitude of infarct expansion, extension, and ventricular remodeling. Other procedures, such as intraaortic balloon pumping, are used to increase the blood pressure driving the coronary blood flow to the areas of the heart at risk adjacent to the infarcted area. These treatments are effective but clearly not satisfactory alone. In many cases, patients arrive at the appropriately equipped hospital too late for these acute therapies. In other cases, their best efforts fail to reopen blood vessels sufficiently to arrest expansion of the infarct. These therapies are also associated with considerable risk to the patient and high cost.

While the methods above attempt to prevent infarction by increase blood/oxygen supply, therapies can also be used to prevent or reduce infarct size by lowering oxygen demand of the heart muscle. In the acute period, pharmaceuticals such as ACE inhibitors, beta-blockers, diuretics, and calcium channel antagonists have the ability to reduce aortic pressure and heart muscle contractility leading to a mild decrease in wall stress. In the chronic post-infarct period, these agents have also been shown to slow the ventricular remodeling process. Nevertheless, in both the acute and chronic periods their ability to reduce the infarct expansion is limited by side effects such as hypotension (pathologically low blood pressure) that can be fatal to a patient.

Also in the chronic period, experimental surgical treatments include approaches to exclude, isolate, or remove the infarct region (such as the Dor procedure). The Dor procedure, also called Endoventricular Patch Plasty, consists in suturing a patch inside the ventricle within the limits of the fibrous scar. Other potential surgical approaches include the application of heat to shrink the infarcted tissue, followed by the suturing of a patch onto the infarcted region. Other experimental treatments envision surrounding the heart, or a significant portion thereof, with a jacket to reduce the size and the wall tension of the heart. However, logistical, surgical and physiological reasons limit the potential use of the techniques to the chronic period. To date, there are no practical, clinically usable, device-based methods of limiting infarct size and expansion by reducing or limiting myocardial wall tension.

The purpose of the heart is to pump blood, thus oxygen and other nutrients, out of the heart to the rest of the body. To accomplish this task, the pressure of the blood in the ventricle of the heart must exceed the pressure in the body's main blood vessel (aorta) leading from the heart. The force needed to generate this increased pressure is created by the contraction of the heart muscle itself. Wall tension can be thought of as a measure of the force by the heart muscle fibers takes into account the ventricular radius at the start of heart muscle contraction. Therefore, when the ventricle needs to generate a greater pressure, for example, with the increased afterload (higher aortic pressure), wall tension is increased. This relationship also shows us that a dilated ventricle (as occurs after an MI or in dilated cardiomyopathy) has to generate increased wall tension to produce the same intraventricular pressure.

Despite spectacular improvements in MI therapy, within one year of the myocardial infarction, 25% of men and 38% of women die. The total number and incidence of heart failure continues to rise with over 500,000 new cases each year. Approximately 85% of these new cases of heart failure are a direct consequence of a large MI. While considerable progress has been made in acute reperfusion of the heart immediately after the MI, heart remodeling and infarct expansion that follows is not treated effectively. There is a clear clinical need for a novel treatment that can be applied shortly after the MI to reduce the extent of the infarct expansion.

### SUMMARY OF THE INVENTION

The invention reduces the severity and complications of MI by reducing infarct size and/or expansion by reducing stress (tension) in the wall of the ventricles of the heart by controllably reducing the amount of blood that fill the ventricles, thus reducing the size (radius) of the ventricle prior to the start of heart contraction. The invention reduces infarct size and/or expansion with a procedure that is practical, simple, easily reversible, and minimally invasive (does not require general anesthesia and surgery).

Multiple animal and human studies have established benefit of reducing arterial blood pressure and cardiac output of the heart in hours and days immediately following MI. The purpose of drugs and devices in the clinical scenario of infarct expansion is the reduction of the myocardial stress and ventricular dilation. The limitation of drugs used for this purpose is that their effect is often too slow, inconsistent, unpredictable and difficult to reverse.

The inventors overcame the limitations of the existing methods and devices for post-MI therapy with a novel and counterintuitive method and technology. The invention limits infarct size and/or expansion by reducing tension in the walls of the heart by temporarily partially occluding parts of the circulatory system such as the great veins that re-fill the heart with blood after each ejection cycle.

It is self evident that the heart can only pump (eject) as much blood as returns to it via the venous system and predominantly via the Inferior Vena Cava (IVC) and to lesser extent via the Superior Vena Cave (SVC) and coronary veins. IVC and SVC converge into the Right Atrium (RA) of the heart. If the amount of venous blood returning to the heart is reduced for example by 10%, the volume and wall stress of the ventricles of the heart, and specifically the left ventricle, will be temporarily reduced allowing heart to heal better and limiting the MI expansion.

In an embodiment of the method of the invention, the amount of venous blood returning to the heart (filling the heart) is reduced by creating a partial temporary obstruction (occlusion) in the IVC or RA. Obstruction can be achieved with an intravascular inflatable balloon placed inside the IVC or RA, or an extravascular occluder cuff placed around the IVC. The inflatable balloon is mounted on a flexible catheter that is similar to “right heart” catheters commonly used by cardiologists to monitor critically ill patients.

The degree of partial occlusion controls the blood flow. As stated above, the reduction in the amount of blood filling the heart will reduced the amount of blood ejected by the heart by the same amount. It is clear that patient safety would be enhanced by providing a method to assure that the device-generated limitation of ventricular filling does not limit blood flow generated by the heart below the level required to maintain adequate vital organ function. The preferred embodiment is equipped with sensors that can measure pressure in the different chambers of the heart, blood flow and oxygen saturation of blood to avoid reducing the blood flow too much. Excessive obstruction of IVC can lead to hypotension (dangerously low blood pressure). Based on these frequent or continuous physiologic measurements the occlusion can be reduced promptly with or without human intervention by an electronic controller mechanism. This feature demonstrates superiority of the invention to conventional drug therapy of MI expansion, since the effect of drugs cannot be accurately predicted or easily reversed.

### SUMMARY OF THE DRAWINGS

A preferred embodiment and best mode of the invention is illustrated in the attached drawings that are described as follows:

FIG. 1 illustrates the right heart catheter equipped with an occlusion balloon placed in the IVC to reduce filling of the heart

FIG. 2 illustrates the treatment of a post-MI patient with the invention

FIG. 3 illustrates the monitoring and control elements of the invention

FIGS. 4 and 5 illustrate embedded software algorithms of the invention

### DETAILED DESCRIPTION OF THE INVENTION

For the proposed clinical use, the capability of the preferred embodiment of the invention is to reduce tension in the walls of the heart by temporarily, controllably and reversibly partially occluding great veins that determine the filling of the heart.

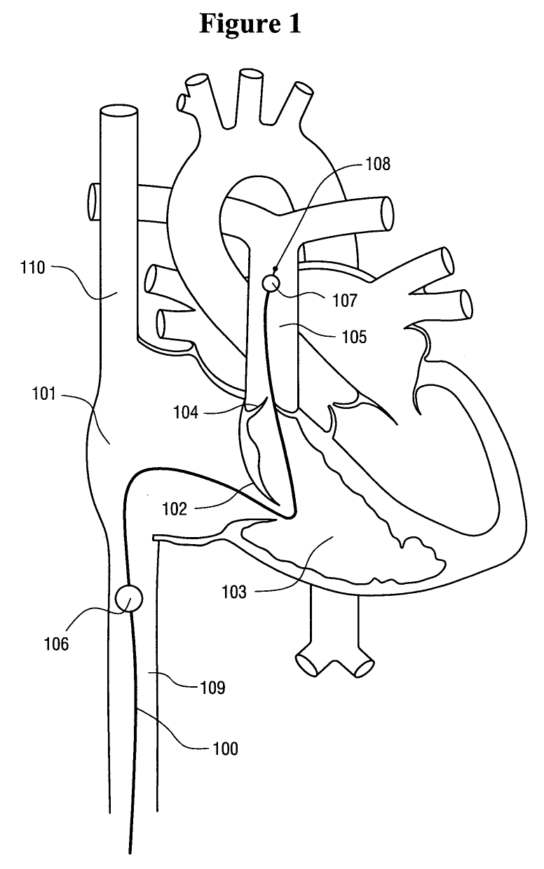

FIG. 1 illustrates one preferred embodiment of the device for this novel treatment that consists of a catheter 100 that is similar to a common Swan-Ganz right heart catheterization catheter. Swan-Ganz catheterization involves the passage of a catheter into the right side of the heart to obtain diagnostic information about the heart and to provide continuous monitoring of heart function in critically ill patients. It has never previously been used or modified to reduce blood flow for treatment of post-MI expansion or any other similar therapy.

During catheterization using a standard Swan-Ganz, a physician inserts the catheter 100 into the right side of the heart through a large vein. Typically, a vein in the right side of the neck is used. However, the left side of the neck, either side of the groin, and other sites can be used. The catheter enters the right atrium 101 (RA or upper chamber) of the heart, flows through the tricuspid valve 102 into the right ventricle 103 (RV or lower chamber), through the pulmonary valve 104, and into the pulmonary artery 105 (PA). Measurements of the pressures in RA, RV, PA and oxygen saturation in the RA or pulmonary artery can be used to indirectly measure the function of the left ventricle. Examples of commercial Swan-Ganz catheters with continuous oxygen saturation monitoring capacity are available from U.S. manufacturers Abbott Laboratories (Opticath) and Edwards Lifesciences (Vigilance CCO/SvO2/CEDV Monitor).

A catheter otherwise similar to these Swan-Ganz catheters is equipped with an additional inflatable, distendable 1 to 8-cc IVC occlusion balloon 106 (further called “occlusion balloon”) located approximately 20 to 30 cm proximally from the conventional distal 1.5-cc PA balloon 107 that is just proximal to the tip 108 that is placed in the pulmonary artery 105. In the preferred embodiment method the catheter 100 is inserted using common femoral vein approach from a puncture in the patient's groin (not shown). During the procedure the occlusion balloon 106 is positioned inside the right atrium (RA) 101, or the inferior vena cava (IVC) 109 preferably using X-ray or fluoroscopic guidance.

FIG. 1 shows the method of obstructing the filling of the heart that the inventors perceived as the most efficient, safe and practical at the time of the invention. An expert in cardiac catheterization can invasion other ways of limiting blood flow to the heart. Specifically it is understood that the occlusion balloon 106, shown in the IVC 109, can be positioned in other places within the right heart and great veins such as in the RA 101, Superior Vena Cava (SVC) 110, right ventricle 103 or pulmonary artery 105 with the similar effect of reducing the filling of the heart. These modifications will not substantially change the invented method, system or device.

Use of catheters to partially occlude blood vessels is known in the field of medical devices. For example, U.S. Pat. No. 6,231,551 to Barbut, incorporated here by reference, and many patents that derive from it describe devices for partial aortic (aorta is the main artery into which the heart ejects oxygenated blood) occlusion for cerebral perfusion (blood flow to the brain) augmentation in patients suffering from ischemia (insufficient oxygen supply). This method has never been previously applied to the right heart and great veins to reduce the tension of the heart wall and to limit infarct expansion. An occlusion device in the aorta, as described by Barbut, will in fact increase the load on the heart and wall tension. It is understood that while the preferred embodiment of this invention uses an inflatable balloon to partially occlude a great vein, other expandable mechanical devices can be envisioned that can be mounted on a catheter and perform the same function.

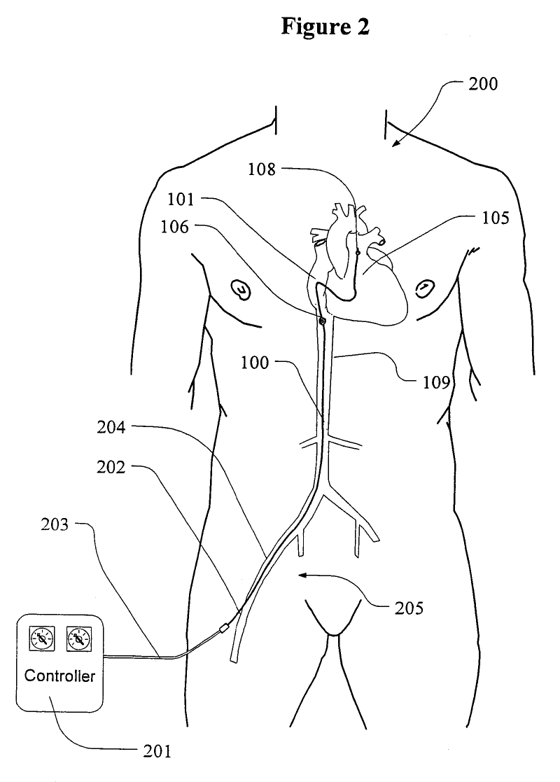

FIG. 2 illustrates the treatment of a post-MI patient 200 with the inventive device. The device basically consists of the vascular catheter 100, inflatable occlusion balloon 106 proximal to the distal tip 108 of the catheter and the controller 201 connected to the proximal end of the catheter 202 by the conduit 203.

Catheter 100 is introduced into the femoral vein 204 of the patient 200 using well-known interventional technique via an incision or puncture in the groin area 205. Catheter has outer diameter of up to 12 French preferably 8 French or less and usable length of 90 to 120 cm. It has multiple internal lumens for inflation of balloons, infusion of drugs and monitoring of blood pressure. Catheter is advanced downstream (towards the heart) into the venous tree into the IVC 109 and further into RA past the right heart valves and into PA 105. Catheter floats into the heart chambers following the flow of blood that carries with it the tip balloon 107. This technique is known in the field of right heart catheterization.

During the insertion and advancement of the catheter stage of the treatment the occlusion balloon 106 is likely deflated and collapsed so as not to interfere with the blood flow. After the position of the balloon 106 is confirmed in the IVC 109 or RA 101 by X-ray, it can be inflated to reduce the blood flow to the heart. The catheter 100 is equipped with radio-opaque markers proximal and/or distal to the balloon 109 to aid visualization and placement.

IVC at the balloon 106 levels is approximately 1.5 to 3 cm in diameter. Therefore, when inflated, balloon 112 shall expand to the diameter of approximately 0.5 to 2.5 cm to effectively partially occlude the IVC. Inflated balloon 106 partially occludes the IVC 109. This creates resistance to blood flow returning to the heart. As a result of this increased resistance stroke volume of the heart is expected to decrease, followed by the desired decrease of diastolic volume of heart ventricles and ventricular wall stress.

Proximal end of the catheter 202 is attached to the control and monitoring console 201 by the flexible conduit 203. Conduit 203 can include balloon inflation lumens and signal-conducting means for monitoring of physiologic variables such as pressures and oxygen saturation. The console 201 includes a microprocessor and sensors and actuators needed to monitor pressures and control the inflation and deflation of the balloon 106.

Integration of physiologic monitoring and right heart catheterization is known. For example an advanced line of Swan Ganz catheters equipped with sensors and corresponding integrated signal processing and patient-monitoring equipment is available from Edwards Lifesciences Corporation (One Edwards Way, Irvine, Calif. 92614). These products available on the U.S. market include:

Edwards Swan-Ganz Continuous Hemodynamic Monitoring Edwards CCO Catheter.

Continuous Cardiac Output (CCO) thermodilution catheters are flow-directed pulmonary artery catheters designed to enable the monitoring of hemodynamic pressures effectively. When used with the Vigilance monitor, CCO catheters allow for continuous calculation and display of cardiac output. The Vigilance monitor used thermal energy emitted by the thermal filament located on the catheter to calculate cardiac output using thermodilution principles.

Edwards CCOmbo Catheter

The Edwards CCOmbo catheter is the abbreviated name for Edwards Swan-Ganz CCO/SVO2/NVIP thermodilution catheters, which are flow-directed pulmonary artery catheters. They are designed to continuously monitor both cardiac output and mixed venous oxygen saturation when used with the Vigilance monitor. Swan-Ganz CCO/SVO2/NVIP thermodilution catheters enable monitoring of hemodynamic pressures and provide an additional (VIP catheter) lumen that allows for continuous infusion. To measure cardiac output continuously, the Vigilance monitor uses thermal energy emitted by the thermal filament located on the catheter to calculate cardiac output using thermodilution principles.

Edwards CCOmbo Volumetrics Catheter

The CCOmbo Volumetrics catheter, the first catheter to offer a continuous EDV (end diastolic volume) measurement, provides the clearest possible picture of hemodynamic performance. The CCOmbo V catheter uses thermodilution and pseudorandom sequencing technologies, enabling clinicians to assess EDV and other volume measurements. Offers complete hemodynamic monitoring with continuous EDV, EF (ejection fraction), SV (stroke volume), SVR, CO and SVO2 parameters. These measurements provide a more reliable indicator of the heart preload than pressure-based measurements.

Edwards Vigilance Monitor

The Edwards Vigilance monitor offers continuous hemodynamic parameters every 60 seconds. CO, SVO2, EDV, EF, SV and SVR parameters are continually displayed on a single display. Calculation and cross-correlation of hemodynamic and oxygenation parameters provide a rapid, comprehensive diagnosis.

Specifically the blood pressure (BP), Continuous Cardiac Output (CCO), SvO2 (Mixed Venous Oxygen Saturation) and continuous EDV (end diastolic volume) measurements available with Edwards Vigilance Monitor technology and similar technologies from other manufacturers can be instrumental to monitor patients undergoing infarct expansion therapy to detect excessive impediment of venous blood flow to the heart and prevent or quickly reverse hypotension.

SvO2 (Mixed Venous Oxygen Saturation) represents the end result of both oxygen delivery and consumption at the tissue level for the entire body. Clinically it can be the earliest indicator of acute deterioration. Sudden decrease of SvO2 is most likely an indication of sudden drop of Cardiac Output—precursor of hypotension. Clinically blood for SvO2 test is drawn from the PA port of the Swan Ganz catheter because it is blood that has been blended in the Right Ventricle. It is a mixture of blood from the Inferior Vena Cava, Superior Vena Cava, and the Coronary Circulation. The catheter 100 can be equipped with miniature SvO2 sensors located at the tip 108. Others sensors located along the shaft of the catheter 100 can include thermistors (for CO measurement by thermodilution) and miniature solid-state pressure sensors. It is reasonable to assume that many new advanced catheter based sensors will become available to designers in future to continuously monitor the performance of the heart. It is understood that such new sensors can be integrated into the current invention in future.

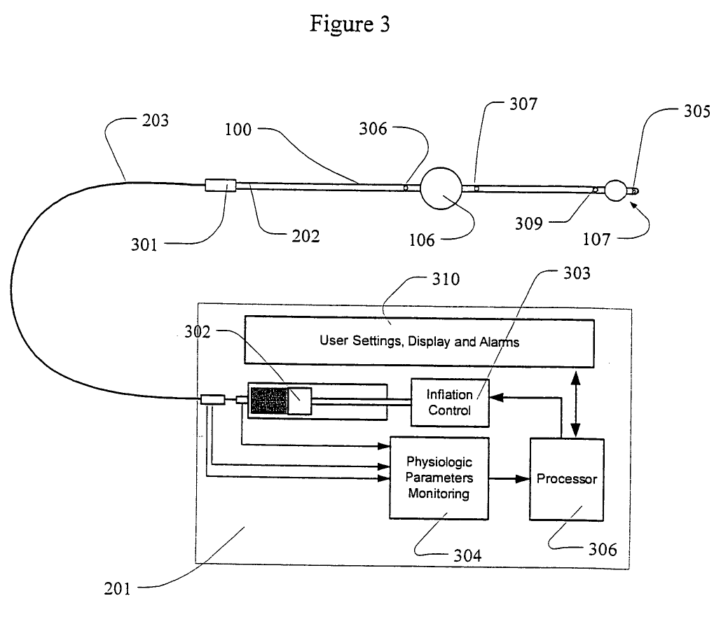

FIG. 3 schematically shows the elements of the preferred embodiment of the invention related to the monitoring of the patient and controlling of the occlusion balloon inflation and deflation.

Catheter 100 is equipped with the balloon 106. Proximal end of the catheter 202 is attached to the control and monitoring console 201 by the flexible conduit 203. Proximal end of the catheter 202 is connected to the flexible conduit 203 with the coupling device 301. The inter-connecting elements between the components of the system are simplified on this drawing. It is understood that different lumens inside the catheter can terminate in separate catheter branches and connect to different receptacles on the console 201. The console itself can consist of several separate modules in separate enclosures.

Controller 201 includes the balloon inflation device 302. Shown in the preferred embodiment is a syringe pump or piston type apparatus. Merit Medical Inc. (South Jordan, Utah) offers a wide variety of these type inflation devices for balloon tipped catheters that can be easily adopted for the invention apparatus. For example Merit Medical manufactures an IntelliSystem® 25 Inflation Syringe for balloon catheters catheter used in cardiology to inflate balloons in coronary arteries of the heart. Alternatively other devices previously used to inflate catheter balloons with compressed gas (such as in Intra-aortic Balloon Pumps) can be used. For example a cylinder with compressed gas under high pressure (not shown) can be connected to the catheter 100 using a pressure regulator and a control valve. Inflation gas can be air, helium or carbon dioxide. Alternatively the balloon 106 can be filled with a liquid such as a radiocontrast agent, saline or water.

Inflation and deflation of the balloon 106 by the inflation device 302 is controlled by the inflation control electronics 303. The inflation control sub-system 303 can include solenoid or other type valves, motors, motor control electronics and common safety features. It is essential that it is able to quickly deflate the balloon 106 by withdrawing the piston 302 or opening a safety valve (not shown) and venting the balloon. The actual design of the balloon inflation sub-system is not essential for the invention and can be implemented using known hydraulic and pneumatic elements.

Controller 201 also includes a monitoring sub-system 304. In the preferred embodiment at least the following physiologic measurements are made: Central Venous Blood Pressure (CVP), Continuous Cardiac Output (CO) and Mixed Venous Blood Oxygen Saturation (SvO2). Sensors integrated with the catheter are used to make actual measurements. For example the SvO2 sensor 305 is sown integrated with the catheter tip 108 for placement in the PA where the venous blood is best mixed. Signals from sensors are transmitted via thin electric wires or fiber optics (not shown) enclosed inside the catheter 100, the conduit 203 and terminate inside the monitoring electronics (sub-system) 304. Advanced micro tip catheter blood pressure transducers (such as ones manufactured by Millar Instruments Inc. Houston, Tex.) can be integrated with the catheter 100 to obtain reliable and accurate measurements of pressure in the RA of the heart 307, in the IVC position 306 or PA position 309 along the catheter. Physiologic signals from the monitoring sub-system 304 are transmitted to the processor 306 that in turn controls the deflation and (optionally) the inflation of the balloon 106 buy controlling the inflation control system 302. The processor 306 can be a microprocessor equipped with software and memory for data storage (not shown). The user interface sub-system 310 is used to display physiologic information to the user and enable the user to set limits for control and safety algorithms embedded in the processor software. For example the user can request the automatic immediate deflation of the balloon 106 if the cardiac output CO of the patient suddenly drops by 20% below the baseline using the user setting keys or other means of system input.

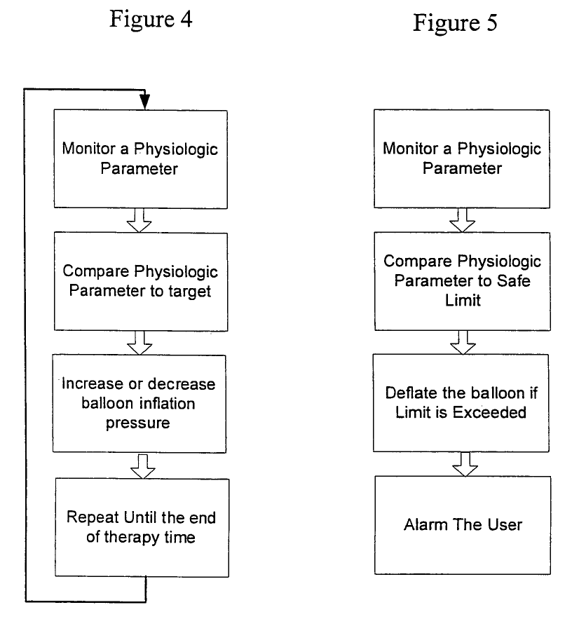

FIG. 4 exemplifies one possible fully automatic algorithm embedded in the software of the controller processor 306. Physiologic parameters indicative of the performance of the patient's heart are monitored continuously and updated as fast as the nature of the particular measurement allows (typically for 5 ms to 60 seconds). The physiologic measurements can include for example: Continuous Cardiac Output (CCO), SvO2 (Mixed Venous Oxygen Saturation), continuous EDV (end diastolic volume), and Central Venous Blood Pressure (CVP). Each one of these parameters can be used as a feedback to control the inflation of the occlusion balloon 106 separately or as a combination index such as a product of CO and SvO2.

Information in digital form is supplied to the processor every 5-10 milliseconds or less frequently if the measurement takes long time. Software algorithm compares the selected parameter to the target values set by the operator or calculated by the processor based on other physiologic information. Algorithm commands the inflation or deflation of the balloon based on these physiologic feedbacks with the objective of achieving the desired safe values set by the physician using the user interface 310. Generally the goal of the algorithm is to achieve the lowest cardiac output that is safe for the particular patient to allow the post-MI heart to heal while operating under minimum stress.

Implementation of the algorithm illustrated by FIG. 4 can be achieved by applying methods known in the field of controls engineering. For example algorithms such as Proportional Integral (PI) controller can be used to maintain a physiologic parameter or calculated index at the target level or within the desired band. Control signals can be applied continuously or periodically to adjust the size of the balloon.

It can be expected that during the therapy the balloon can stretch, leak gas or that the patient's condition such as the cardiac contractility, heart rate and peripheral vascular resistance can change. In response to these changes the balloon size (defined by pressure or volume of the infused fluid) may require a correction. It can be envisioned that the operator, based on the readings of physiologic sensors, can make the correction manually. An automatic response has advantage of saved time and increased safety but makes the system more complex and expansive.

FIG. 5 illustrates a less sophisticated algorithm that relies on the operator intervention to implement the post-MI therapy. The size balloon is adjusted manually to achieve the desired levels of cardiac performance. The software monitors the physiologic parameters for signs of hypotension. If a sign of hypotension such as a sudden drop or slow deterioration of SvO2 or CO is detected the balloon is rapidly deflated and the obstruction to blood flow is removed. The user is notified by the alarm and can restart therapy after the patient is stabilized.

The proposed system does not need to depend on expansive integrated catheter based measurements. Both invasive (such as thermo dilution) and non invasive (such as bio-impedance) methods of measuring cardiac offer similar physiologic controls that may be vital for patients with weakened hearts. In both cases a decrease of the cardiac output will indicate that the balloon is impeding the ejection of the heart too much and shall be deflated. Both invasive and non-invasive physiologic measurements are well known in the practice of medicine and can be implemented separately or in combination in an integrated system or by connecting the inventive device to existing clinical monitors present in the Intensive Care Units of any modern hospital.

The invention has been described in connection with the best mode now known to the applicant inventors. The invention is not to be limited to the disclosed embodiment. Rather, the invention covers all of various modifications and equivalent arrangements included within the spirit and scope of the appended claims. Common to all the embodiments is that the flow of blood to the heart is partially impeded by obstruction of great vessels to reduce the wall tension of the heart and allow it to heal after the acute MI. The obstruction is controlled based on physiologic parameters to avoid excessive reduction of blood flow. Treatment can be rapidly reversed at any time by removing the obstruction.

What is claimed is:

1. A method for treating an infarct of a heart in a human patient comprising:
inserting a catheter into a vein of the patient, wherein the catheter comprises a proximal region, a distal region and an expandable member mounted on the distal region;
advancing the catheter through the vein and into a vena cava of the patient;
locating the expandable member in an inferior vena cava (IVC) or a right atrium (RA) of the patient; and
expanding the expandable member to partially occlude flow of venous blood into the heart, wherein blood flow through the heart is decreased and stress on a wall of the heart is decreased.

2. The method as in claim 1 where the expandable member is a balloon.

3. The method as in claim 1 further comprising measuring a physiologic parameter of the patient indicative of the heart performance and adjusting the expansion of the expandable member based on the measured parameter.

4. The method as in claim 3 where the physiologic parameter is chosen from the group comprising Cardiac Output (CO), SvO2 (Mixed Venous Oxygen Saturation), continuous EDV (end diastolic volume), and Blood Pressure (BP).

5. A medical device for partial occlusion of an IVC of a patient comprising:
a catheter further comprising proximal and distal ends, wherein the distal end is adapted for insertion through a peripheral vein, a inferior vena cava, a right atrium, a tricuspid valve, the right ventricle, a pulmonary valve and into a pulmonary artery, and
an expandable balloon at the distal end of the elongate member, the balloon expandable to partially occlude inferior vena cava or right atrium to reduce blood flow to the heart.

6. The device of claim 5 further comprising at least one physiologic sensor mounted distal the expandable balloon for measuring a parameter indicative of the performance of the heart.

7. The device of claim 5 further comprising a variable balloon size expansion mechanism for controlling blood flow to the heart.

8. The device of claim 6 wherein the physiologic sensor is chosen from a group consisting of: Cardiac Output (CO), SvO2 (Mixed Venous Oxygen Saturation), and Blood Pressure (BP) sensors.

9. The device of claim 6 further wherein said expandable balloon reduces in response to a change of the parameter based on a signal from the physiologic sensor.
